(19) United States
(12) Patent Application Publication
(10) Pub. No.: US 2009/0175942 A1
(43) Pub. Date: Jul. 9, 2009

(54) Solid Dosage Form of Olmesartan Medoxomil And Amlodipine

(75) Inventors: Wolfgang Bauer, Pfaffenhofen (DE); Johann Lichey, Pfaffenhofen (DE); Andreas Teubner, Pfaffenhofen (DE); Elmar Wadenstorfer, Pfaffenhofen (DE)

Correspondence Address: KING & SPALDING, 1185 AVENUE OF THE AMERICAS, NEW YORK, NY, 10036-4003, US

(73) Assignee: DAIICHI SANKYO COMPANY, LIMITED, Tokyo (JP)

(21) Appl. No.: 12/401,748

(22) Filed: Mar. 11, 2009

Related U.S. Application Data
(63) Continuation of application No. PCT/GB2007/003933, filed on Oct. 12, 2007.

(51) Int. Cl. A61K 9/32; A61K 31/4422; A61K 31/546; A61K 9/28; A61P 9/12

(52) U.S. Cl. 424/482; 514/356; 514/222.5; 424/474

(57) ABSTRACT

The invention relates to a stable solid dosage form comprising olmesartan medoxomil and amlodipine or a pharmacologically acceptable salt thereof. In particular, it relates to solid dosage forms free from reducing sugars. The stable solid dosage form may optionally further comprise hydrochlorothiazide or a pharmacologically acceptable salt thereof.

This application claims priority under 35 U.S.C. § 120 as a continuation from co-pending application PCT/GB2007/003933 filed Oct. 12, 2007, which is hereby incorporated by reference in its entirety.

### TECHNICAL FIELD OF THE INVENTION

The present invention relates to a solid dosage form comprising olmesartan medoxomil and amlodipine and optionally further comprising hydrochlorothiazide.

### BACKGROUND OF THE INVENTION

Olmesartan medoxomil is an angiotensin II receptor antagonist developed for the treatment of hypertension and other medical indications as disclosed in U.S. Pat. No. 5,616,599. Its chemical name is 2,3-dihydroxy-2-butenyl 4-(1-hydroxy-1-methylethyl)-2-propyl-1-[p-(o-1H-tetrazol-5-ylphenyl)benzyl]imidazole-5-carboxylate, cyclic 2,3-carbonate or (5-methyl-2-oxo-1,3-dioxolen-4-yl)methyl 4-(1-hydroxy-1-methylethyl)-2-propyl-1-{4-[2-(tetrazol-5-yl)phenyl]phenyl}methylimidazole-5-carboxylate having the following structure:

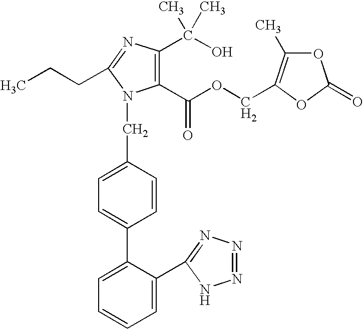

Olmesartan medoxomil is marketed by Sankyo under the trade name of Olmetec® or Benicar®. It is available as oral tablets in strengths of 5 mg, 10 mg, 20 mg and 40 mg. The inactive ingredients in the Olmetec® tablets include low-substituted hydroxypropylcellulose, microcrystalline cellulose, lactose monohydrate, hydroxypropylcellulose and magnesium stearate.

Olmesartan medoxomil is a prodrug which, after ingestion, liberates the only active metabolite, 4-(1-hydroxy-1-methylethyl)-2-propyl-1-[[2′-(1H-tetrazol-5-yl)biphenyl-4-yl]methyl]-1H-imidazol-5-carboxylic acid (RNH-6270). The chemical structure of RNH-6270 is:

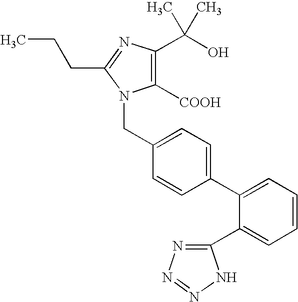

Under acidic or basic conditions and in the presence of water, RNH-6270 is formed by hydrolysis of the ester bond of olmesartan medoxomil.

Amlodipine is a calcium channel blocker developed for the treatment of hypertension and other medical indications as disclosed in U.S. Pat. No. 4,572,909 and U.S. Pat. No. 4,879,303. Its chemical name is 3-ethyl-5-methyl-(±)-2-[(2-aminoethoxy)methyl]-4-(2-chlorophenyl)-1,4-dihydro-6-methylpyridine-3,5-dicarboxylate, having the following structure:

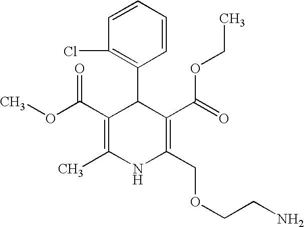

Amlodipine is marketed by Pfizer as the monobenzenesulfonate salt, amlodipine besylate under the trade name Norvasc®. It is available as oral tablets in strengths of 2.5 mg, 5 mg and 10 mg. The inactive ingredients in the Norvasc® tablets include microcrystalline cellulose, dibasic calcium phosphate anhydrous, sodium starch glycolate and magnesium stearate.

WO 2006/059217 discloses that amlodipine is highly hygroscopic and absorbs moisture, which leads to degradation. One of the major routes of degradation is via a catalytic oxidative process, which is pH dependent. One of the major degradation products is 3-ethyl-5-methyl-2-[(2-aminoethoxy)methyl]-4-(2-chlorophenyl)-6-methylpyridine-3,5-dicarboxylate (Impurity D). The chemical structure of Impurity D is:

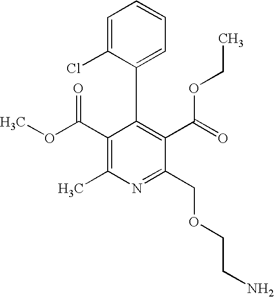

Pharmaceutical Development and Technology, vol. 9, No. 1, pp. 15-24, 2004 discloses that mixtures of lactose, basic excipients and water induce some instability in amlodipine besylate because of a Maillard reaction between the primary amino group and lactose.

As amlodipine is an unstable compound, well-directed approaches are required to formulate pharmaceutical compositions with reasonable stability.

Although WO 04/067003 and EP 1604664 disclose a medicament comprising olmesartan medoxomil and amlodipine, there is no known stable solid dosage form comprising olmesartan medoxomil and amlodipine.

### OBJECTS OF THE INVENTION

The mechanisms of action of olmesartan medoxomil and amlodipine are believed to cooperate favorably in the treatment or prophylaxis of hypertension or diseases caused by hypertension. As this assumption becomes supported by an increasing amount of clinical data, there is an escalating need for a fixed dose combination drug comprising the active ingredients olmesartan medoxomil and amlodipine. However, both olmesartan medoxomil and amlodipine are chemical compounds that are difficult to formulate owing to stability problems of said active ingredients. Therefore, although there is a clear need for a fixed dose combination drug which combines the features of adequate drug stability and solubility with pharmacological efficacy, to achieve this a number of technical problems must be overcome. It is an object of the present invention to provide such a fixed dose combination drug.

There are various types of solid dosage forms that could be considered, but it cannot be predicted which of these dosage forms combines product stability, solubility and pharmacological efficacy in the best manner. Generally, a fixed-dose combination of drugs intended for instant release is prepared by making a powder mixture of a co-granulate of the two active ingredients with the necessary excipients, by keeping the basic formulation of one of the corresponding mono-drug preparations and simply adding the second drug component.

With a combination of olmesartan medoxomil and amlodipine, this approach does not appear feasible due to the incompatibility of amlodipine with components of the conventional olmesartan medoxomil formulations. When an Olmetec® based formulation is used for the fixed dose combination drug, degradation products appear in the dosage form because of a Maillard reaction between amlodipine and lactose in the formulation. When a Norvasc® based formulation is used, on the other hand, solubility and bioavailability of the olmesartan medoxomil decreases. Furthermore, the preparations of olmesartan medoxomil and amlodipine currently on the market have several drawbacks. The weights of the known Olmetec® tablets and Norvasc® tablets are relatively high (218 mg and 432 mg in Olmetec® tablets, 200 mg and 400 mg in Norvasc® tablets, respectively). Due to the large amount of the excipients present in the formulations, the tablet size for both the Olmetec® and Norvasc® formulations is relatively large, and such large tablets are difficult to swallow, especially for aged patients. The present invention is directed towards the preparation of a stable solid dosage form comprising olmesartan medoxomil and amlodipine which overcomes the aforementioned problems.

### SUMMARY OF THE INVENTION

The object of the present invention is to provide a solid dosage form comprising olmesartan medoxomil and amlodipine or a pharmacologically acceptable salt thereof with improved stability of the active ingredients and reduced weight. In accordance with the present invention, problems associated with the preparation of a solid dosage form comprising olmesartan medoxomil and amlodipine or a pharmacologically acceptable salt thereof can best be handled by means of the preparation of formulations that are substantially free of reducing sugar in the formulation.

The present invention provides solid dosage forms comprising olmesartan medoxomil and amlodipine or a pharmacologically acceptable salt thereof, which are characterized by having less than 2.5% concentration (w/w) of RNH-6270, less than 0.4% concentration (w/w) of Impurity D and less than 5.1% concentration (w/w) of total impurities and by being substantially free of reducing sugar (particularly a dosage form for the prophylaxis or treatment of hypertension), the use of olmesartan medoxomil and amlodipine or a pharmacologically acceptable salt thereof to manufacture the aforementioned solid dosage form (particularly a dosage form for the prophylaxis or treatment of hypertension), a method for preventing or treating a disease (particularly hypertension) in which the aforementioned solid dosage form comprising pharmacologically effective amounts of olmesartan medoxomil and amlodipine or a pharmacologically acceptable salt thereof is administered to warm-blooded animals (particularly humans) and a use of a solid dosage form comprising olmesartan medoxomil and amlodipine or a pharmacologically acceptable salt thereof in the manufacture of a medicament for preventing or treating a disease (particularly hypertension). In a preferred embodiment of the invention, the solid dosage form of the invention further comprises the thiazide diuretic hydrochlorothiazide, which has the following structural formula:

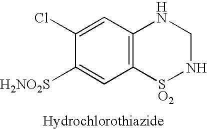

Specifically, the present invention provides:

(1) A solid dosage form comprising olmesartan medoxomil and amlodipine or a pharmacologically acceptable salt thereof, having less than 2.5% concentration (w/w) of 4-(1-hydroxy-1-methylethyl)-2-propyl-1-[[2′-(1H-tetrazol-5-yl)biphenyl-4-yl]methyl]-1H-imidazol-5-carboxylic acid (RNH-6270).
(2) A solid dosage form comprising olmesartan medoxomil and amlodipine or a pharmacologically acceptable salt thereof, having less than 0.4% concentration (w/w) of 3-ethyl-5-methyl-2-[(2-aminoethoxy)methyl]-4-(2-chlorophenyl)-6-methylpyridine-3,5-dicarboxylate (Impurity D).
(3) A solid dosage form comprising olmesartan medoxomil and amlodipine or a pharmacologically acceptable salt thereof, having less than 5.1% concentration (w/w) of total impurities.
(4) A solid dosage form comprising olmesartan medoxomil and amlodipine or a pharmacologically acceptable salt thereof, having less than 2.5% concentration (w/w) of RNH-6270 and less than 5.1% concentration (w/w) of total impurities.
(5) A solid dosage form according to (1) or (2), further comprising hydrochlorothiazide or a pharmacologically acceptable salt thereof.
(6) A solid dosage form according to (5), having less than 7.3% concentration (w/w) of total impurities.
(7) A solid dosage form comprising olmesartan medoxomil and amlodipine or a pharmacologically acceptable salt thereof, wherein said solid dosage form is substantially free of reducing sugars.
(8) A solid dosage form according to (1), wherein said solid dosage form is substantially free of a reducing sugars.
(9) A solid dosage form according to (2), wherein said solid dosage form is substantially free of reducing sugars.
(10) A solid dosage form according to (3), wherein said solid dosage form is substantially free of reducing sugars.
(11) A solid dosage form according to (4), wherein said solid dosage form is substantially free of reducing sugars.
(12) A solid dosage form according to (5) or (6), wherein said solid dosage form is substantially free of reducing sugars.
(13) A solid dosage form according to any one of (7) to (12), wherein said solid dosage form has less than 2.0% (w/w) of reducing sugars.
(14) A solid dosage form according to any one of (7) to (12), wherein said solid dosage form has less than 0.3% (w/w) of reducing sugars.
(15) A solid dosage form according to any one of (7) to (12), wherein said solid dosage form has less than 0.05% (w/w) of reducing sugars.
(16) The solid dosage form according to any one of (1), (5) and (7) to (15) having less than 0.5% concentration (w/w) of RNH-6270.
(17) The solid dosage form according to any one of (1), (5) and (7) to (15) having less than 0.4% concentration (w/w) of RNH-6270.
(18) The solid dosage form according to any one of (2), (5) and (7) to (15), having less than 0.3% concentration (w/w) of Impurity D.
(19) The solid dosage form according to any one of (2), (5) and (7) to (15), having less than 0.05% concentration (w/w) of Impurity D.
(20) The solid dosage form according to any one of (3) and (5) to (15), having less than 1.5% concentration (w/w) of total impurities.
(21) The solid dosage form according to any one of (4) to (15), having less than 0.5% concentration (w/w) of RNH-6270 and less than 1.5% concentration (w/w) of total impurities.
(22) The solid dosage form according to any one of (4) to (15), having less than 0.4% concentration (w/w) of RNH-6270 and less than 1.5% concentration (w/w) of total impurities.
(23) The solid dosage form according to any one of (1) to (6) and (16) to (22) wherein the concentration of said impurity or impurities is that measured after accelerated testing of said solid dosage form for three months at 40° C. and 75% relative humidity.
(24) The solid dosage form according to any one of (1) to (23) wherein the amlodipine is present in the form of its besylate salt.
(25) The solid dosage form according to any one of (1) to (24), further comprising one or more pharmacologically acceptable additives.
(26) The solid dosage form according to (25), wherein the one or more pharmacologically acceptable additives are selected from excipients, lubricants, binders, disintegrants, emulsifiers, stabilizers, correctives and diluents.
(27) The solid dosage form according to (26), wherein the excipient is silicified microcrystalline cellulose and/or mannitol.
(28) The solid dosage form according to (26), wherein the lubricant is magnesium stearate.
(29) The solid dosage form according to (26), wherein the disintegrant is pregelatinised starch and/or croscarmellose sodium.
(30) The solid dosage form according to any one of (1) to (29), wherein the solid dosage form comprises a tablet.
(31) The solid dosage form according to (30), wherein the tablet is prepared by direct compression.
(32) The solid dosage form according to (30) or (31) wherein the tablet is coated with at least one elastic film.
(33) The solid dosage form according to (32), wherein the elastic film contains at least one hydrophilic polymer.
(34) The solid dosage form according to (33), wherein the hydrophilic polymer is polyvinyl alcohol and/or macrogol.
(35) The solid dosage form according to any one of (1) to (34), comprising 20 to 40 mg of olmesartan medoxomil.
(36) The solid dosage form according to any one of (1) to (35), comprising 5 to 10 mg of amlodipine or a pharmacologically acceptable salt of amlodipine equivalent to 5 to 10 mg of amlodipine.
(37) The solid dosage form according to any one of (1) to (36), comprising 12.5 to 25 mg of hydrochlorothiazide or a pharmacologically acceptable salt of hydrochlorothiazide equivalent to 12.5 to 25 mg of hydrochlorothiazide.
(38) A method for the treatment or prophylaxis of hypertension in a warm-blooded animal in need thereof, comprising administering to said animal an effective amount of a solid dosage form according to any one of (1) to (37).
(39) Use of a solid dosage form according to any one of (1) to (37) in the manufacture of a medicament for the treatment or prophylaxis of hypertension.
(40) A solid dosage form according to any one of (1) to (37) for use in the treatment or prophylaxis of hypertension.

### BRIEF DESCRIPTION OF DRAWINGS

FIG. 1 shows the results for the concentration of Impurity D and RNH-6270 as measured in Test Example 1 for Olmetec®, Norvasc®, the formulation of Example 1 and the formulation of Reference Example 1.

FIG. 2 shows the results for the rates of dissolution for the formulation of Example 1 and the formulation of Reference Example 1 as measured in Test Example 2.

### DETAILED DESCRIPTION OF THE INVENTION

The solid dosage form of the present invention contains olmesartan medoxomil and amlodipine or a pharmacologically acceptable acid salt thereof as its active ingredients, and optionally further contains hydrochlorothiazide or a pharmacologically acceptable acid salt thereof.

Olmesartan medoxomil can easily be produced according to the methods disclosed in the art, suitable examples including the methods disclosed in U.S. Pat. No. 5,616,599.

Amlodipine can be easily produced according to the methods disclosed in the art, suitable examples including the methods disclosed in U.S. Pat. No. 4,572,909. Amlodipine can be used as a pharmacologically acceptable acid salt thereof, such as a besylate, maleate, fumarate, camsylate, hydrochloride, hydrobromide, lactate, tartrate, citrate, mesylate, nicotinate, gluconate and the like, as well as in the form of a free base. Of these, amlodipine besylate is preferably used.

Hydrochlorothiazide can be easily produced according to the methods disclosed in the art, suitable examples including the methods disclosed in U.S. Pat. No. 3,025,292. The compound name of hydrochlorothiazide is 6-chloro-3,4-dihydro-2H-1,2,4,-benzothiadiazin-7-sulfonamide 1,1-dioxide. The hydrochlorothiazide of this invention includes pharmacologically acceptable salts thereof, for example, a hydrohalogenic acid salt such as hydrofluoride, hydrochloride, hydrobromide or hydroiodide; nitrate; perchlorate; sulfate; phosphate; a C₁-C₄ alkanesulfonic acid salt, which may be optionally substituted with a halogen atom(s) such as methanesulfonate, trifluoromethanesulfonate or ethanesulfonate; a C₆-C₁₀ arylsulfonic acid salt, which may be optionally substituted with a C₁-C₄ alkyl group(s), such as benzenesulfonate or p-toluenesulfonate; a C₁-C₆ aliphatic acid salt such as acetate, malate, fumarate, succinate, citrate, tartrate, oxalate or maleate; or an amino acid salt such as the glycine salt, lysine salt, alginine salt, ornitine salt, glutamic acid salt or aspartic acid salt. The preferred salts are the hydrochloride, nitrate, sulfate or phosphate and the particularly preferred salt is hydrochloride.

In one aspect of the invention, the solid dosage form has less than 2.5% concentration (w/w), preferably less than 0.5% concentration (w/w), and more preferably less than 0.4% concentration (w/w) of RNH-6270. In another aspect of the invention, the solid dosage form also has less than 0.4% concentration (w/w), preferably less than 0.3% concentration (w/w) and more preferably less than 0.05% concentration (w/w) of Impurity D. In yet another aspect, the solid dosage form also has less than 5.1% concentration (w/w), and preferably less than 1.5% concentration (w/w) of total impurities.

In one preferred aspect, the solid dosage form further comprises hydrochlorothiazide or a pharmacologically acceptable salt thereof. In a preferred aspect of this triple combination solid dosage form (comprising olmesartan medoxomil, amlodipine or a pharmacologically acceptable salt thereof and hydrochlorothiazide or a pharmacologically acceptable salt thereof), the solid dosage form has less than 2.5% concentration (w/w), preferably less than 0.5% concentration (w/w), and more preferably less than 0.4% concentration (w/w) of RNH-6270. In another preferred aspect of the triple combination solid dosage form of the invention, the solid dosage form also has less than 0.4% concentration (w/w), preferably less than 0.3% concentration (w/w) and more preferably less than 0.05% concentration (w/w) of Impurity D. In yet another aspect, the triple combination solid dosage form also has less than 7.3% concentration (w/w), and preferably less than 1.5% concentration (w/w) of total impurities.

The term “stable” as used herein refers to chemical stability of olmesartan medoxomil and/or amlodipine or a pharmacologically acceptable acid salt thereof in the solid dosage forms and indicates the presence of less than 2.5% concentration (w/w) of RNH-6270 and/or less than 0.4% concentration (w/w) of Impurity D and/or less than 5.1% concentration (w/w) of total impurities. For solid dosage forms of the invention further comprising hydrochlorothiazide or a pharmacologically acceptable salt thereof, the term “stable” as used herein refers to chemical stability of olmesartan medoxomil and/or amlodipine or a pharmacologically acceptable acid salt thereof in the solid dosage forms and indicates the presence of less than 2.5% concentration (w/w) of RNH-6270 and/or less than 0.4% concentration (w/w) of Impurity D and/or less than 7.3% concentration (w/w) of total impurities. Preferably, the stability is measured using HPLC to measure the presence of related substances after accelerated testing for three months at 40° C. and 75% relative humidity on the basis of the percentage concentrations of the impurities relative to the active substances from which they are derived, e.g. a 2.5% concentration (w/w) of RNH-6270 means that at the time of measuring, the amount of RNH-6270 is 2.5% of the amount of olmesartan medoxomil as measured at the same time. This stability data is provided below in Table 1, in terms of the percent concentrations (w/w) relative to the active substances from which they are derived.

The term “total impurities” as used herein refers to the total degradation products derived from olmesartan medoxomil and amlodipine or a pharmacologically acceptable salt thereof. Where the solid dosage form further comprises hydrochlorothiazide or a pharmacologically acceptable salt thereof, the “total impurities” also include degradation products derived from said hydrochlorothiazide or a pharmacologically acceptable salt thereof.

A reducing sugar is a type of sugar with an aldehyde group, which allows the sugar to act as a reducing agent, for example in a Maillard reaction or a Benedict's reaction. Examples of “reducing sugars” include, but are not limited to, lactose, glucose, fructose, glyceraldehyde, arabinose, mannose, galactose, maltose, xylose, cellobiose, mellibiose, maltotriose, and the like, as well as hydrates thereof.

The term “substantially free” as used herein refers to the use of a reducing sugar in a concentration less than is suitable for it to be used as an excipient. The solid dosage form preferably has less than 2.0% (w/w) of reducing sugars, more preferably less than 0.3% (w/w) of reducing sugar and most preferably less than 0.05% (w/w) reducing sugars.

The solid dosage form of the present invention can where desired additionally contain at least one further additive such as a suitable pharmacologically acceptable excipient, lubricant, binder, disintegrants, emulsifier, stabilizer, corrective or diluent.

Suitable “excipients” include, but are not limited to, either individually or in combination, organic excipients including non-reducing sugar derivatives such as sucrose, trehalose, mannitol or sorbitol; starch derivatives such as corn starch, potato starch, α-starch or dextrin; cellulose derivatives such as microcrystalline cellulose or silicified microcrystalline cellulose; gum Arabic; dextran; and pullulan, and inorganic excipients including silicate derivatives such as light anhydrous silicic acid, synthetic aluminum silicate, calcium silicate or magnesium metasilicate aluminate; phosphates such as dibasic calcium hydrogenphosphate or calcium hydrogen phosphate dihydrate; carbonates such as calcium carbonate; and sulfates such as calcium sulfate. Of these, silicified microcrystalline cellulose and mannitol are preferably used.

Suitable “lubricants” include, but are not limited to, either individually or in combination, stearic acid; stearic acid metal salts such as calcium stearate or magnesium stearate; talc; colloidal silica; waxes such as beeswax or spermaceti; boric acid; adipic acid; sulfates such as sodium sulfate; glycol; fumaric acid; sodium benzoate; D,L-leucine; lauryl sulfates such as sodium lauryl sulfate or magnesium lauryl sulfate; silicates such as silicic anhydride or silicate hydrate; and the aforementioned starch derivatives. Of these, magnesium stearate is preferably used.

Suitable “binders” include, but are not limited to, either individually or in combination, hydroxypropyl cellulose, hydroxypropyl methyl cellulose, polyvinylpyrrolidone, macrogol and compounds similar to the aforementioned excipients.

Suitable “disintegrants” include, but are not limited to, either individually or in combination, cellulose derivatives such as low-substituted hydroxypropyl cellulose, carboxymethyl cellulose, calcium carboxymethyl cellulose or internally crosslinked sodium carboxymethyl cellulose; cross-linked polyvinylpyrrolidone; and chemically modified starches/celluloses such as carboxymethyl starch, sodium carboxymethyl starch, sodium starch glycolate, pregelatinised starch or croscarmellose sodium. Of these, pregelatinised starch and croscarmellose sodium are preferably used.

Suitable “emulsifiers” include, but are not limited to, either individually or in combination, colloidal clays such as bentonite or bee gum; metal hydroxides such as magnesium hydroxide or aluminum hydroxide; anionic surfactants such as sodium lauryl sulfate or calcium stearate; cationic surfactants such as benzalkonium chloride; and nonionic surfactants such as polyoxyethylene alkyl ether, polyoxyethylene sorbitan fatty acid ester or sucrose fatty acid ester.

Suitable “stabilizers” include, but are not limited to, either individually or in combination, para-hydroxybenzoic acid esters such as methyl paraben or propyl paraben; alcohols such as chlorobutanol, benzyl alcohol or phenyl ethyl alcohol; benzalkonium chloride; phenols such as phenol or cresol; thimerosal; dehydroacetic acid; and sorbic acid.

Suitable “correctives” include, but are not limited to, either individually or in combination, sweeteners such as sodium saccharin or aspartame; sour flavourings such as citric acid, malic acid or tartaric acid; and fragrances such as menthol, lemon or orange fragrance.

Suitable “diluents” include, but are not limited to, either individually or in combination, mannitol, sucrose, calcium sulfate, calcium phosphate, hydroxypropyl cellulose, microcrystalline cellulose, water, ethanol, polyethylene glycol, propylene glycol, glycerol, starch, polyvinylpyrrolidone, magnesium metasilicate aluminate, and mixtures thereof.

The “solid dosage form” of the present invention comprises any dosage form used by the person skilled in the art to deliver one or more pharmacologically active ingredients to a patient in a solid form. Suitable solid dosage forms will be well known to the person skilled in the art, and non-limiting examples of the solid dosage form of the present invention include tablets (including sublingual tablets and tablets that disintegrate in the mouth), capsules (including soft capsules and microcapsules), granules, pills and lozenges. Of these, tablets are most preferred.

A solid dosage form of the present invention may be produced using any commonly used method well known to a person skilled in the art of pharmaceutical formulation technology and there are no particular limitations thereon. Examples of suitable methods include those disclosed in publications such as Powder Technology and Pharmaceutical Processes [D. Chulia et al., Elsevier Science Pub. Co. (Dec. 1, 1993)].

A tablet of the present invention can be obtained by a direct compression method. In a direct compression method, the active ingredients, together with one or more pharmacologically acceptable additives, are blended in a suitable blender, then transferred directly to a compression machine for pressing into a tablet. Other conventional methods such as wet granulation or dry granulation can also be used.

In addition, a tablet of the present invention may also be provided with at least one layer of a film coating. If a film coating is desired, any film coating apparatus of a type well known in the art can be used, and as film coating bases, suitable examples include sugar coating bases, hydrophilic film coating bases, enteric film coating bases and sustained release film coating bases.

Suitable examples of sugar coating bases include saccharose, and these can be used in combination with one or more additives such as talc, precipitated calcium carbonate, calcium phosphate, calcium sulfate, gelatin, gum Arabic, polyvinylpyrrolidone and pullulan.

Suitable examples of hydrophilic film coating bases include cellulose derivatives such as hydroxypropyl cellulose, hydroxypropyl methyl cellulose (e.g., Opadryl® OY S 38956 (white), commercially available from Colorcon, Inc.), hydroxyethyl cellulose, methyl hydroxyethyl cellulose and sodium carboxymethyl cellulose; synthetic polymers such as polyvinyl acetal diethyl aminoacetate, aminoalkyl methacrylate copolymer, polyvinylpyrrolidone, polyvinyl alcohol (e.g., Opadryl® II, commercially available from Colorcon, Inc.), polyvinylalcol-polyethylene glycol graft-copolymers (e.g., Kollicoat® IR, commercially available from BASF) and macrogol; and polysaccharides such as pullulan. Of these, polyvinyl alcohol and macrogol are preferably used.

Suitable examples of enteric film coating bases include cellulose derivatives such as hydroxypropyl methyl cellulose, phthalate hydroxypropyl methyl cellulose acetate succinate, carboxymethyl ethyl cellulose and cellulose acetate phthalate; acrylic acid derivatives such as methacrylic acid copolymer L, methacrylic acid copolymer LD and methacrylic acid copolymer S; and natural substances such as shellac.

Suitable examples of sustained release film coating bases include cellulose derivatives such as ethyl cellulose; and acrylic acid derivatives such as aminoalkyl methacrylate copolymer RS, ethyl acrylate-methyl methacrylate copolymer emulsion.

A mixture of two or more different coating bases such as those above may also be used in a suitable ratio. In addition, the coating films may also contain suitable pharmacologically acceptable additives such as plasticizers, excipients, lubricants, opacifying agents, colorants or antiseptics as necessary.

The doses and the dosing ratios of olmesartan medoxomil and amlodipine or a pharmacologically acceptable salt thereof and, where applicable, hydrochlorothiazide or a pharmacologically acceptable salt thereof, which are the active ingredients in the solid dosage form of the present invention, can be changed depending on various factors such as the activity of each of the active ingredients and the symptoms, age and body weight of the patient. Although the dosage varies depending on symptoms, age and the like, in the case of oral administration, the dosage of olmesartan medxomil is typically from 5 mg to 80 mg, preferably 10 to 40 mg per day, the dosage of amlodipine or a pharmacologically acceptable salt thereof is typically equivalent to from 2.5 mg to 20 mg, preferably 5 to 10 mg per day of amlodipine, and the dosage of hydrochlorothiazide or a pharmacologically acceptable salt thereof is typically equivalent to from 5 mg to 50 mg, preferably 12.5 to 25 mg per day of hydrochlorothiazide for a human adult. The dosage can be administered from one to six times, preferably one time, per day depending on the symptoms of the patients.

In addition, the dosing ratio of olmesartan medoxomil and amlodipine or a pharmacologically acceptable salt thereof, which are the active ingredients in the solid dosage form of the present invention, can also be changed over a wide range. For example, the dosing ratio by weight of olmesartan medoxomil and amlodipine or a pharmacologically acceptable salt thereof can be typically within a range of 1:50 to 50:1, preferably within a range of 1:5 to 5:1. Presently, preferred forms are tablets comprising 40/10 mg, 40/5 mg, 20/10 mg, 20/5 mg, 10/10 mg and 10/5 mg of olmesartan medoxomil and amlodipine or a pharmacologically acceptable salt thereof equivalent to said amount of amlodipine, respectively. For the triple combination further comprising hydrochlorothiazide or a pharmacologically acceptable salt thereof, the dosing ratio by weight of olmesartan medoxomil, amlodipine or a pharmacologically acceptable salt thereof and hydrochlorothiazide or a pharmacologically acceptable salt thereof can be typically within a range of 1:50:1-50 to 50:1-50, preferably within a range of 1:5:1-5 to 5:1:1-5. Presently, preferred forms are tablets comprising 40/10/12.5 mg, 40/5/12.5 mg, 40/10/25 mg, 40/5/25 mg, 20/10/12.5 mg and 20/5/12.5 mg of olmesartan medoxomil, amlodipine or a pharmacologically acceptable salt thereof equivalent to said amount of amlodipine and hydrochlorothiazide or a pharmacologically acceptable salt thereof equivalent to said amount of hydrochlorothiazide, respectively.

The total weight of the solid dosage form containing olmesartan medoxomil and amlodipine or a pharmacologically acceptable salt thereof as the sole active agents, containing 40 mg of olmesartan medoxomil amounts to 100 mg to 300 mg, preferably to about 200 mg. The total weight of the solid dosage form containing olmesartan medoxomil and amlodipine or a pharmacologically acceptable salt thereof as the sole active agents, containing 20 mg of olmesartan medoxomil amounts to 50 mg to 150 mg, preferably to about 100 mg. The total weight of the triple combination solid dosage form containing olmesartan medoxomil, amlodipine or a pharmacologically acceptable salt thereof and hydrochlorothiazide or a pharmacologically acceptable salt thereof, containing 40 mg of olmesartan medoxomil amounts to 100 mg to 400 mg, preferably to about 300 mg.

The solid dosage form of the present invention is effective for the prophylaxis or treatment of, for example, hypertension or diseases caused by hypertension [more specifically, hypertension, heart disease (angina pectoris, myocardial infarction, arrhythmia, cardiac insufficiency or hypercardia), kidney disease (diabetic nephropathy, glomerular nephritis or nephrosclerosis), or cerebrovascular disease (cerebral infarction or cerebral hemorrhage)] and the like.

### EXAMPLES

The present invention will be described in more detail by way of the following examples, but the scope of the present invention is not limited thereto.

### Example 1

Composition of a tablet:

| | |
|---|---|
| Olmesartan medoxomil | 40.00 mg |
| Amlodipine besylate | 13.89 mg |
| Pregelatinized starch | 70.00 mg |
| Silicified microcrystalline cellulose | 65.31 mg |
| Croscarmellose sodium | 10.00 mg |
| Magnesium stearate | 0.80 mg |
| Opadry ® II | 8.00 mg |
| Total weight | 208.00 mg |

Tablets were prepared according to the composition listed above using the following steps.

The powder mixture was prepared in a tumbling blender by mixing the active ingredients (milled olmesartan medoxomil and amlodipine besylate) with pregelatinized starch, silicified microcrystalline cellulose and croscarmellose sodium.

The powder mixture was then screened, using a screening mill, with a 1.9 mm screen. The screened powder mixture was blended again in a tumbling blender.

Magnesium stearate was added to the powder mix and blended in the tumbling blender to produce the final blend. The final blend was compressed into slightly convex tablets using a rotary press, the size and shape appropriate to the tablet strength.

The coating suspension was prepared by dispersing Opadryl® II in purified water. The tablet cores underwent a film-coating procedure using standard coating equipment.

### Example 2

Composition of a tablet:

| | |
|---|---|
| Olmesartan medoxomil | 40.00 mg |
| Amlodipine besylate | 13.89 mg |
| Hydrochlorothiazide | 12.50 mg |
| Pregelatinized starch | 105.00 mg |
| Silicified microcrystalline cellulose | 112.41 mg |
| Croscarmellose sodium | 15.00 mg |
| Magnesium stearate | 1.20 mg |
| Opadry ® II | 10.00 mg |
| Total weight | 310.00 mg |

Tablets were prepared according to the composition listed above using the following steps.

The powder mixture was prepared in a tumbling blender by mixing the active ingredients (milled olmesartan medoxomil, amlodipine besylate and hydrochlorothiazide) with pregelatinized starch, silicified microcrystalline cellulose and croscarmellose sodium.

The powder mixture was then screened, using a screening mill, with a 1.9 mm screen. The screened powder mixture was blended again in a tumbling blender.

Magnesium stearate was added to the powder mix and blended in the tumbling blender to produce the final blend. The final blend was compressed into slightly convex tablets using a rotary press, the size and shape appropriate to the tablet strength.

The coating suspension was prepared by dispersing Opadryl® II in purified water. The tablet cores underwent a film-coating procedure using standard coating equipment.

### Reference Example 1

### Olmetec® Based Formulation

Composition of a tablet:

| | |
|---|---|
| Olmesartan medoxomil | 40.00 mg |
| Amlodipine besylate | 13.89 mg |
| Low-substituted hydroxypropylcellulose | 80.00 mg |
| Microcrystalline cellulose | 40.00 mg |
| Lactose monohydrate | 232.51 mg |
| Hydroxypropylcellulose | 10.00 mg |
| Magnesium stearate | 3.60 mg |
| Opadry ® OY S 38956 | 12.00 mg |
| Total weight | 432.00 mg |

Tablets were prepared according to the composition listed above using the following steps.

The powder mixture was prepared in a wet high-shear granulator by mixing the active ingredients (milled olmesartan medoxomil, amlodipine besylate) with low-substituted hydroxypropylcellulose, microcrystalline cellulose, lactose monohydrate and hydroxypropylcellulose and then kneaded with purified water.

The wet granules were screened, using a screening mill, with a 9.5 mm screen, and then dried in a fluid bed dryer.

The dried granules were screened, using a screening mill, with a 1.9 mm screen.

Magnesium stearate was added to the screened granules and blended in the tumbling blender to produce the final blend.

The final blend was compressed into slightly convex tablets using a rotary press, the size and shape appropriate to the tablet strength.

The coating suspension was prepared by dispersing Opadryl® OY S 38956 (white) in purified water. The tablet cores underwent a filmcoating procedure using standard coating.

### Test Example 1

### Storage Stability Test

The tablets of Example 1 to be tested were put into HDPE bottles with desiccant, and the bottles were closed tightly with a HDPE screw. The tablets in the bottles were stored at 40° C. under 75% R.H. (the accelerated test) for 3 months.

Impurities derived from degradation of olmesartan medoxomil and amlodipine in the tablets were determined by HPLC (Agilent 1100 systems, Agilent Technologies Co., Ltd.). The results were as follows:

TABLE 1

| | | | Reference |
|---|---|---|---|
| | Olmetec ® + Norvasc ® | Example 1 | Example 1 |
| RNH-6270 | 0.57 | 0.38 | 0.46 |
| Impurity D | 0.31 | 0.04 | 0.04 |
| Total | — | 0.87 | 1.55 |
| impurities | | | |

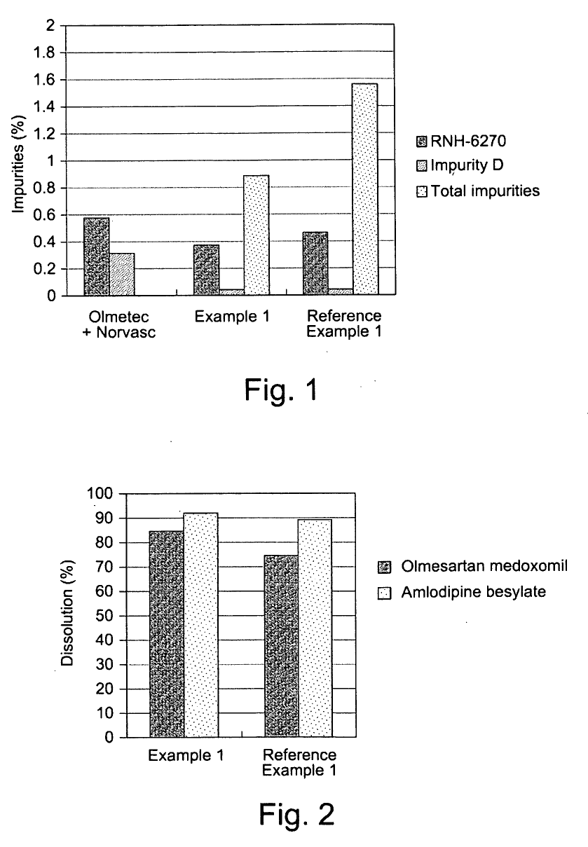

As can be seen in Table 1 and FIG. 1, the formulation of Example 1, a formulation of the present invention demonstrated superior stability compared to olmesartan medoxomil and amlodipine formulations commercially available as Olmetec® and Norvasc®, respectively.

Based on the results presented in Table 1 and FIG. 1, a correlation can also be seen between the formation of impurities and the presence or absence of reducing sugars in the formulation. Reference Example 1, which had lactose in the formulation, had a relatively high level of total impurities after three months. In contrast, the formulation of Example 1 was substantially free of reducing sugars in the formulation and consequently had a significantly lower level of total impurities relative to Reference Example 1.

Thus, the data in Table 1 and FIG. 1 indicate that the stability of dosage forms comprising olmesartan medoxomil and amlodipine can be improved depending on the presence or absence of reducing sugars in the formulation.

### Test Example 2

### Dissolution Test

For dissolution testing of a tablet of Example 1, an EP/USP dissolution tester equipped with a diode array spectrophotometer, suitable for Multi-component-Analysis (MCA) was used.

The key parameters are as follows:

Medium: Phosphate buffer solution pH 6.8+/−0.5 (Jap. Pharm)

Volume: 900+/−9 mL

Temperature: 37.0+/−0.5° C.

Bath type: USP apparatus 2

Stirrer: 50 rpm+/−2 rpm

The amounts of olmesartan medoxomil and amlodipine besylate dissolved were determined by Multi-Component-Analysis (MCA) of filtered portions of the solution under test in comparison with respective reference solutions.

TABLE 2

| | Example 1 - | Reference Example 1 - |
|---|---|---|
| | Dissolution (%) | Dissolution (%) |
| Olmesartan medoxomil | 84.0 | 74.0 |
| Amlodipine besylate | 91.7 | 89.4 |

As can be seen in Table 2 and FIG. 2, the formulation of Example 1 demonstrated superior dissolution properties for both olmesartan medoxomil and amlodipine besylate compared to the formulation of Reference Example 1.

### Test Example 3

### Storage Stability Test

The tablets of Example 2 to be tested were put into HDPE bottles with desiccant, and the bottles were closed tightly with a HDPE screw. The tablets in the bottles were stored at 40° C. under 75% R.H. (the accelerated test) for 3 months.

Impurities derived from degradation of olmesartan medoxomil, amlodipine and hydrochlorothiazide in the tablets at the end of the 3 month period were determined by HPLC (Agilent 1100 systems, Agilent Technologies Co., Ltd.). The results were as follows:

TABLE 3

| | Olmetec ® + Norvasc ® | Example 1 | Example 2 |
|---|---|---|---|
| RNH-6270 | 0.57 | 0.38 | 0.34 |
| Impurity D | 0.31 | 0.04 | <0.04 |
| Total | | 0.87 | 0.57 |
| impurities | | | |

As can be seen in Table 3, the formulation of Example 2, a triple combination formulation of the present invention demonstrated superior stability compared to olmesartan medoxomil and amlodipine formulations commercially available as Olmetec® and Norvasc®, respectively, with significantly lower levels of RNH-6270 and Impurity D even after accelerated testing for 3 months. The triple combination formulation of the present invention showed excellent stability; indeed, it can be seen from the above comparison that the stability was even a little higher than that for the double combination product of the present invention tested in Test Example 1.

### Test Example 4

### Dissolution Test

For dissolution testing of a tablet of Example 2, an EP/USP dissolution tester equipped with a diode array spectrophotometer, suitable for Multi-component-Analysis (MCA) was used.

The key parameters are as follows:

Medium: Phosphate buffer solution pH 6.8+/−0.5 (Jap. Pharm)

Volume: 900+/−9 mL

Temperature: 37.0+/−0.5° C.

Bath type: USP apparatus 2

Stirrer: 50 rpm+/−2 rpm

The amounts of olmesartan medoxomil, amlodipine besylate and hydrochlorothiazide dissolved were determined by Multi-Component-Analysis (MCA) of filtered portions of the solution under test in comparison with respective reference solutions. The results from Test Example 2 above are included for comparison.

TABLE 4

| | Reference | Example 1 | Example 2 |
|---|---|---|---|
| Olmesartan medoxomil | 74.0 | 84.0 | 82.0 |
| Amlodipine besylate | 89.4 | 91.7 | 90.0 |
| Hydrochlorothiazide | | | 99.0 |

As can be seen in Table 4, the formulation of Example 2 demonstrated excellent dissolution properties for olmesartan medoxomil, amlodipine besylate and hydrochlorothiazide.

On the basis of the above experiments, it can be readily determined that both the quality and the stability of tablets of Examples 1 and 2 prepared according to the present invention are fully satisfactory.

### INDUSTRIAL APPLICABILITY

According to the present invention, a stable solid dosage form comprising olmesartan medoxomil and amlodipine or a pharmacologically acceptable salt thereof, and optionally comprising hydrochlorothiazide is obtained.

1. A solid dosage form comprising olmesartan medoxomil and amlodipine or a pharmacologically acceptable salt thereof, having less than 2.5% concentration (w/w) of 4-(1-hydroxy-1-methylethyl)-2-propyl-1-[[2′-(1H-tetrazol-5-yl)biphenyl-4-yl]methyl]-1H-imidazol-5-carboxylic acid (RNH-6270).

2. A solid dosage form comprising olmesartan medoxomil and amlodipine or a pharmacologically acceptable salt thereof, having less than 0.4% concentration (w/w) of 3-ethyl-5-methyl-2-[(2-aminoethoxy)methyl]-4-(2-chlorophenyl)-6-methylpyridine-3,5-dicarboxylate (Impurity D).

3. A solid dosage form comprising olmesartan medoxomil and amlodipine or a pharmacologically acceptable salt thereof, having less than 5.1% concentration (w/w) of total impurities.

4. A solid dosage form comprising olmesartan medoxomil and amlodipine or a pharmacologically acceptable salt thereof, having less than 2.5% concentration (w/w) of RNH-6270 and less than 5.1% concentration (w/w) of total impurities.

5. A solid dosage form according to claim 1 or claim 2, further comprising hydrochlorothiazide or a pharmacologically acceptable salt thereof.

6. A solid dosage form according to claim 5, having less than 7.3% concentration (w/w) of total impurities.

7. A solid dosage form comprising olmesartan medoxomil and amlodipine or a pharmacologically acceptable salt thereof, wherein said solid dosage form is substantially free of reducing sugars.

8. A solid dosage form according to claim 1, wherein said solid dosage form is substantially free of a reducing sugars.

9. A solid dosage form according to claim 2, wherein said solid dosage form is substantially free of reducing sugars.

10. A solid dosage form according to claim 3, wherein said solid dosage form is substantially free of reducing sugars.

11. A solid dosage form according to claim 4, wherein said solid dosage form is substantially free of reducing sugars.

12. A solid dosage form according to claim 5 or claim 6, wherein said solid dosage form is substantially free of reducing sugars.

13. A solid dosage form according to any one of claims 7 to 12, wherein said solid dosage form has less than 2.0% (w/w) of reducing sugars.

14. A solid dosage form according to any one of claims 7 to 12, wherein said solid dosage form has less than 0.3% (w/w) of reducing sugars.

15. A solid dosage form according to any one of claims 7 to 12, wherein said solid dosage form has less than 0.05% (w/w) of reducing sugars.

16. The solid dosage form according to any one of claims 1, 5 and 7 to 15 having less than 0.5% concentration (w/w) of RNH-6270.

17. The solid dosage form according to any one of claims 1, 5 and 7 to 15 having less than 0.4% concentration (w/w) of RNH-6270.

18. The solid dosage form according to any one of claims 2, 5 and 7 to 15, having less than 0.3% concentration (w/w) of Impurity D.

19. The solid dosage form according to any one of claims 2, 5 and 7 to 15, having less than 0.05% concentration (w/w) of Impurity D.

20. The solid dosage form according to any one of claims 3 and 5 to 15, having less than 1.5% concentration (w/w) of total impurities.

21. The solid dosage form according to any one of claims 4 to 15, having less than 0.5% concentration (w/w) of RNH-6270 and less than 1.5% concentration (w/w) of total impurities.

22. The solid dosage form according to any one of claims 4 to 15, having less than 0.4% concentration (w/w) of RNH-6270 and less than 1.5% concentration (w/w) of total impurities.

23. The solid dosage form according to any one of claims 1 to 6 and 16 to 22 wherein the concentration of said impurity or impurities is that measured after accelerated testing of said solid dosage form for three months at 40° C. and 75% relative humidity.

24. The solid dosage form according to any one of claims 1 to 23 wherein the amlodipine is present in the form of its besylate salt.

25. The solid dosage form according to any one of claims 1 to 24, further comprising one or more pharmacologically acceptable additives.

26. The solid dosage form according to claim 25, wherein the one or more pharmacologically acceptable additives are selected from excipients, lubricants, binders, disintegrants, emulsifiers, stabilizers, correctives and diluents.

27. The solid dosage form according to claim 26, wherein the excipient is silicified microcrystalline cellulose and/or mannitol.

28. The solid dosage form according to claim 26, wherein the lubricant is magnesium stearate.

29. The solid dosage form according to claim 26, wherein the disintegrant is pregelatinised starch and/or croscarmellose sodium.

30. The solid dosage form according to any one of claims 1 to 29, wherein the solid dosage form comprises a tablet.

31. The solid dosage form according to claim 30, wherein the tablet is prepared by direct compression.

32. The solid dosage form according to 30 or claim 31 wherein the tablet is coated with at least one elastic film.

33. The solid dosage form according to claim 32, wherein the elastic film contains at least one hydrophilic polymer.

34. The solid dosage form according to claim 33, wherein the hydrophilic polymer is polyvinyl alcohol and/or macrogol.

35. The solid dosage form according to any one of claims 1 to 34, comprising 20 to 40 mg of olmesartan medoxomil.

36. The solid dosage form according to any one of claims 1 to 35, comprising 5 to 10 mg of amlodipine or a pharmacologically acceptable salt of amlodipine equivalent to 5 to 10 mg of amlodipine.

37. The solid dosage form according to any one of claims 1 to 36, comprising 12.5 to 25 mg of hydrochlorothiazide or a pharmacologically acceptable salt of hydrochlorothiazide equivalent to 12.5 to 25 mg of hydrochlorothiazide.

38. A method for the treatment or prophylaxis of hypertension in a warm-blooded animal in need thereof, comprising administering to said animal an effective amount of a solid dosage form according to any one of claims 1 to 37.

39. Use of a solid dosage form according to any one of claims 1 to 37 in the manufacture of a medicament for the treatment or prophylaxis of hypertension.

40. A solid dosage form according to any one of claims 1 to 37 for use in the treatment or prophylaxis of hypertension.
